(19) United States
(12) Patent Application Publication
(10) Pub. No.: US 2008/0082047 A1
(43) Pub. Date: Apr. 3, 2008

(54) Vein holder

(76) Inventor: Stoney Race Harmon, Manchester, TN (US)

Correspondence Address: Stoney Race Harmon, 184 Hendrixson Dr, Manchester, TN, 37355, US

(21) Appl. No.: 11/509,545

(22) Filed: Aug. 24, 2006

(51) Int. Cl. A61M 5/00

(52) U.S. Cl. 604/115

(57) ABSTRACT

A vein holder to prevent vein roll during vein puncture procedures while also preventing the caregiver from accidentally sticking themselves. The legs of the vein holder are to be adjusted as needed to straddel the size of vein being used. Once placed on the caregiver's thumb and the legs adjusted to the proper width apart, the vein holder is placed on the patient's skin, just over the chosen vein, and pressed down lightly into the patient's skin to hold the vein from rolling left or right. The caregiver's other hand holds the needle and then punctures the vein between the legs of the vein holder. If the needle misses it's mark, for any reason, the caregiver will not stick their own thumb by accident.

### BACKGROUND OF THE INVENTION

In medicine, few procedures are more common than vein punctures. Phlebotomies, intravenous injections, and intravenous drip and heparin lock insertions are all prolifically practiced. In many patients, however, vein punctures have a high rate of failure.

In older patients, for example, vein hardening and a decrease in the underlying connective tissue that holds veins in place increases the tendency of veins to roll during vein punctures. Additionally, pediatric, trauma and mentally ill patients all present high rates of vein puncture failure. A device that quickly, safely and easily holds veins in place would significantly reduce vein puncture failures.

Some phlebotomists try to decrease such failures by straddling a patient's vein with their fingernails. Positioned thusly, they hold a vein in place and puncture it successfully on the first try. However, using this method, they sometimes accidentally puncture their own person. Other phlebotomists seek to decrease vein puncture failures by manually pulling tight the skin about a vein in an attempt to stabilize the underlying vein.

In view of the foregoing, it is one object of the instant invention to provide a phlebotomy device which reduces complications, such as rolling veins, inherent with vein punctures. It is another object of the invention to provide a phlebotomy device which reduces the risk of unwanted needle punctures by shielding the thumb of the caregiver, which holds a blood vessel in place. Another object of the invention is to teach a device for which both ease and speed of use allows phlebotomists to puncture a patient's blood vessel without altering their present phlebotomy techniques.

These and other objects of the present invention will become readily apparent upon further review of the following specification and drawings.

### SUMMARY OF THE INVENTION

A device for quickly, safely, and easily holding veins in place during vein punctures is disclosed. A body fabricated of rigid or semi-rigid material such as plastic or metal fits about a caregiver's thumb. Fitted thusly, the vein holder device is placed upon a patient's skin, just over a chosen vein, and pressed down lightly. On one end of the device's body, at the portion furthest from the caregiver's thumb, is a pair of lips. A first stationary lip rests snugly along the side of the chosen vein closest to the phlebotomist. A second, moveable lip, sits on the opposite side of this chosen vein. This second lip may be adjusted as needed to straddle the chosen vein. With the chosen vein thus locked into position, the caregiver's free hand guides a needle between the vein holder lips, until the chosen vein is punctured.

### BRIEF DESCRIPTION OF THE DRAWINGS

The structure, operation and advantages of the presently preferred embodiment of this invention will become further apparent upon consideration of the following description, taken in conjunction with the accompanying drawings, wherein:

FIG. 1 is a side view of my new vein holder showing the adjustable second lip detached and ready to insert into the front slit of the main body.

FIG. 2 is a side, partial cut-away, view of a vein holder showing the adjustable second lip inserted through the slit into the internal hollow space of the main body.

FIG. 3 is a side view of a vein holder, shown on a caregiver's left thumb, illustrating how to adjust the second lip of the device.

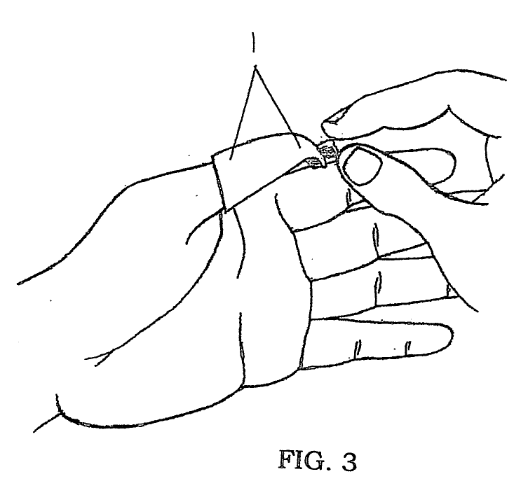

FIG. 4 is a side view of a vein holder, illustrating both lips straddling a chosen vein, with the second lip locked in place, and the caregiver's free hand set to puncture the vein.

### DETAILED DESCRIPTION OF THE DRAWINGS

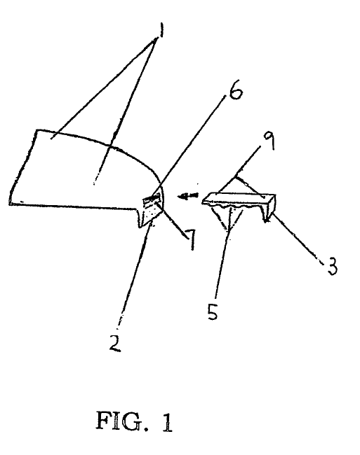

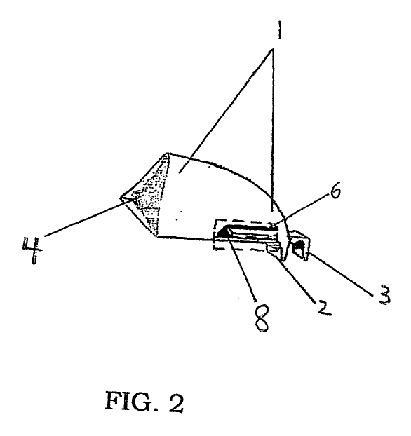

Referring to FIG. 1 and by way of example only, one embodiment of Applicant's vein holder is shown. Here, the device consists of a body, 1, and an adjustable second lip, 3. An exemplary construction of second lip, 3, by way of example, is shown in FIG. 1. In general, the second lip, 3, is L shaped, with the underside of its stalk, 9, characterized by a series of ridges, 5. FIG. 2 illustrates the insertion of second lip, 3, into slit, 6. Directly behind slit, 6, is an internal hollow space, 8. Referring to FIG. 2, this internal hollow space, 8, is roughly equal in height and depth to the stalk, 9, of the L shaped second lip, 3.

As second lip, 3, is passed into slit, 6, and threaded into internal hollow space, 8, any of the second lip ridges, 5, may catch upon a catch lip, 7, thus halting forward progress of second lip, 3. Forward progress can be halted at any desired ridge, 5, along the underside of second lip, 3, thus locking second lip, 3, at a desirable distance from first lip, 2, in order to adjust a gap between the lips to accommodate the width of a vein to be punctured.

As my vein holder is intended to be fitted generally about the girth of a user's thumb, another feature of the invention, as seen in FIG. 2, opening, 4, is provided for this function. Because the hollow interior of body 1 tapers as it moves from the opening, 4, toward first lip, 2, and because body, 3, may be constructed of semi rigid material such as metal or plastic, the vein holder may be adjusted to fit snugly about a large variety of thumbs.

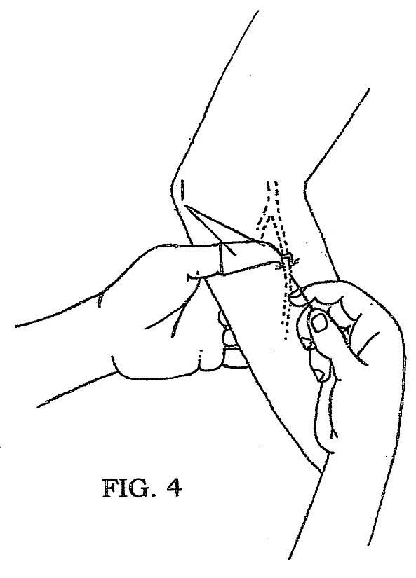

Once placed about a user's thumb, as seen in FIG. 4, the vein holder may be set against a chosen arm and the first and second lips, 2 and 3 respectively, may be adjusted and locked about an intended vein. FIG. 4 further illustrates how the vein holder, once engaged, allows a secondary hand complete freedom for holding and maneuvering a syringe or blood draw implement. Also, as noted, construction of my device is of metal or plastic so as to protect a user's digit against inadvertent needle sticks.

Having thus described my invention and the manner of its use, it should be apparent to those of relevant skill in the arts to which my invention pertains that incidental changes may be made thereto that fairly fall within the scope of the following appended claims, wherein I claim:

1. (canceled)

2. (canceled)

3. (canceled)

4. (canceled)

5. (canceled)

6. (canceled)

7. (canceled)

8. (canceled)

9. A medical device for securing a blood vessel for puncture and for protecting a digit of a caregiver or user from accidental needle sticks, said medical device comprising:
a generally hollow, elongated body of a rigid or semi-rigid material having an opening at one end for receiving at least a portion of a thumb of a user,
a pair of lips mounted at an opposite end of said hollow body, said lips defining a gap therebetween that may be adjusted to accommodate a width of said blood vessel to be punctured whereby said blood vessel is prevented from movement while being pierced.

10. A medical device as set forth in claim 9, wherein an end of said hollow body opposite said opening is provided with a tapered end, with said pair of lips disposed on said tapered end.

11. A medical device as set forth in claim 10, wherein said pair of lips is oriented normal to an axis of said elongated hollow body.

12. A medical device as set forth in claim 11, wherein one of said pair of lips is attached to a stalk member movable into and out of said tapered end.

13. A medical device as set forth in claim 12 wherein said stalk member and said tapered end are configured to lock said one of said pair of lips in a selected position, to define a width of said gap engaging said blood vessel.

14. A medical device as set forth in claim 13 wherein said stalk member and said tapered end further comprises:
a plurality of ridges on said stalk portion,
a catch lip in said tapered end of said hollow elongated body whereby valleys between said plurality of ridges engage said catch lip to lock said one of said pair of lips so that said gap is held to a width of said blood vessel.

15. A medical device for facilitating a blood vessel puncture comprising:
a generally hollow body having an opening at one end for receiving a digit of a user,
a first, stationary lip on an opposite end of said body, said lip extending generally normal to said digit and toward a blood vessel to be punctured,
a second lip on an opposite end of said body and extending generally normal to said digit, and further disposed in a plane parallel to said first lip, said second lip movable generally along an axis of said digit so as to adjust a gap between said first lip and said second lip, said gap being adjustable so as to accommodate a width of a blood vessel and hold the blood vessel in a fixed position beneath the skin of a patient so that the blood vessel may be pierced without rolling,
said first lip and said second lip also being of a length so as to secure the blood vessel along said length thereof.

16. A medical device as set forth in claim 15, wherein said generally hollow body is fabricated of a material so as to resist needle sticks.

17. A medical device as set forth in claim 15, wherein said second lip further comprises a plurality of catches for locking said second lip in place in an adjusted position.

18. A medical device as set forth in claim 15, wherein said body further comprises a slit on an opposite end of said body from said opening, said slip adapted for accepting said catches to lock said second lip in place.

19. A medical device as set forth in claim 18, wherein the lower edge of said slit is configured to hold said catches for locking said second lip in said adjusted position.

20. A medical device as set forth in claim 15 wherein the generally hollow body is configured according to the body of claim 10.

21. A medical device for facilitating a blood vessel puncture comprising:
a generally hollow body, tapering at one end to a tapered end,
an opening at an opposite end of said hollow body for receiving a digit of a user, said hollow body configured so as to snugly fit about digits of variable girth,
a first, stationary lip on said opposite end of said body, said lip extending generally normal to said digit and toward a blood vessel to be punctured,
a second lip on said opposite end of said body and extending generally normal to said digit, and further disposed in a plane parallel to said first lip, said second lip attached to a stalk portion perpendicular to said second lip, said stalk portion movably extending into said opposite end of said body, said first lip and said second lip defining a gap therebetween,
said first lip and said second lip approximately of a width of said tapered end of said body,
said stalk portion characterized by a plurality of ridges,
said tapered end of said body having a hollow, stalk-receiving space for receiving said stalk portion characterized by said plurality of said ridges, said hollow generally equal in length to said stalk of said second lip, said stalk portion slideably insertable into said hollow stalk-receiving space,
said first and second lip also being of a length of said tapered end so as to secure the blood vessel along a length thereof, whereby said gap may be adjustable to a fixed position by moving said stalk portion into or out of said hollow, stalk-receiving space so as to accommodate a width of a blood vessel and hold the blood vessel in a fixed position beneath the skin of a patient so that the blood vessel may be pierced without rolling,

22. A medical device as set forth in claim 21, wherein said generally hollow body is made of rigid or semi-rigid material, so as to resist inadvertent and accidental needle sticks to a user.

23. A medical device as set forth in claim 21, wherein said stalk portion and said second lip are detachable from said generally holow body.
